(19) United States
(12) Patent Application Publication
(10) Pub. No.: US 2015/0072369 A1
(43) Pub. Date: Mar. 12, 2015

(54) INSECT-BASED MODEL FOR TESTING THE EFFECTS OF NANOPARTICLES ON THE FUNCTION AND INTEGRITY OF THE BLOOD-BRAIN BARRIER

(71) Applicant: ENTOMOPHARM APS, Copenhagen OE (SE)

(72) Inventors: Peter Aadal Nielsen, Oxie (SE); Gunnar Andersson, Roestaanga (SE); Olga Andersson, Roestaanga (SE)

(21) Appl. No.: 14/389,067

(22) PCT Filed: Mar. 15, 2013
(86) PCT No.: PCT/DK2013/050075

§ 371 Date: Sep. 29, 2014

(30) Foreign Application Priority Data
Mar. 29, 2012 (DK) PA 2012 00232

(51) Int. Cl. G01N 33/50

(52) U.S. Cl. CPC G01N 33/5085; G01N 2500/04
USPC 435/29

(57) ABSTRACT

There is provided a method and model insects for screening the effects of nanoparticles on brain barrier function and integrity. The method involves exposing the insect brain-barrier to the nanoparticle(s) of interest and exposing the nanoparticle treated insect brain barrier to one or more suitable marker(s) of the function and integrity of the brain barrier.

### FIELD OF THE INVENTION

The present invention is directed to insect models that are aimed to reflect the effect of nanoparticles on the vertebrate blood-brain barrier (BBB) function and integrity. Specifically, the present invention relates to the use of insects in assessing potential safety issues of nanoparticles at the blood-brain barrier.

### BACKGROUND OF THE INVENTION

Although there are anecdotal data indicating a causal relationship between long-term ultrafine particle exposures in ambient air (e.g., traffic related) or at the workplace (e.g., metal fumes) and resultant neurotoxic effects in humans, more studies are needed to test the hypothesis that inhaled nanoparticles (NP) or NPs absorbed via food cause neurodegenerative effects or CNS functional defects. Some NPs may have a significant toxicity (hazard) potential, and this will pose a significant risk if there is a sufficient exposure while others may have a more long term deleterious effect on cell and organ function including CNS. The challenge is to identify such hazardous NPs and take appropriate measures to prevent exposure.

It has been shown that certain NPs do permeate the BBB and in this relation it is important that the NPs are readily cleared from the brain such that the NPs do not cause any brain damage or functional disturbances. Hindering the NP from entering the brain is not straight forward since the mechanism describing how the NPs permeate the BBB still is under debate. However, it is of utmost importance to identify NPs that permeate or affect the function of the BBB in order to address potential safety issues.

Among the non-invasive approaches, polymeric nanoparticles, especially poly(butylcyanoacrylate) (PBCA) nanoparticles coated with polysorbate 80, have recently received much attention from neuroscientists as an attractive and innovative carrier for brain targeting. These nanoparticles may be defined as “a submicron drug-carrier system”, which are generally polymeric in nature. Since nanoparticles are small in size, they easily penetrate into small capillaries and can be taken up within cells, allowing efficient drug accumulation at targeted sites in the body. The first reported nanoparticles were based on non-biodegradable polymeric systems. Their use for systemic administration, however, could not be considered because of the possibility of chronic toxicity due to the tissue and immunological response towards the non-biodegradable polymer. Hence, nanoparticles prepared from biodegradable polymers such as poly(cyanoacrylate) were exclusively studied. The use of biodegradable materials for nanoparticle preparation allows sustained drug release at the targeted site over a period of days or even weeks after injection.

Since the use of NPs has increased tremendously during the recent years and the NPs are widely spread also in the environment and there are great concerns for the potential effects on the function of living organisms as well as human beings.

Investigation of the effects of NPs on BBB function and integrity is extremely important since there is an increasing use of nanoparticles and the safety profile for many of these are yet to be understood. Moreover, nanoparticles are important in drug discovery as they have proven to be useful as carriers for potential CNS drugs.

Certain insects may be suitable as model organisms for studying the effect of NPs on the BBB function and integrity. Insects are multi cell organisms with complex compartmentalized nervous systems for specialized functions like vision, olfaction, learning, and memory. The nervous system of the insects responds physiologically in similar ways as in vertebrates with many identical neurohormones and receptors. Insects have avascular nervous systems in which hemolymph bathes all outer surfaces of ganglia and nerves. Therefore, many insects require a sophisticated brain barrier (BB) system to protect their CNS from plant-derived neurotoxins and to maintain an appropriate ionic microenvironment of the neurons. In fact, also in insects a sophisticated BB system has been an evolutionary advantage. In insects this BB is mainly based on the glia cell system which certainly shifted to the endothelial system in the vertebrate brain as a response to an increased importance on a microvasculature system. In support of this view is the appearance of the glia system in elasmobranch fish and the remnants of their glia barrier in modern mammalian CNS. Thus, insects possess a BB which is an important component in the ensheathment of the nervous system. The BBs in insects are highly sophisticated but varies in structure between different insect orders. Thus, insects with highly sophisticated brain barriers with complex integrative components that mimic the vertebrate barriers will be excellent models for documentation of the effect of NPs in insects and prediction of these effects on the BBB function and integrity of vertebrates including man.

There is an obvious need for efficient screening of the effect of NPs on the BBB in order to address the safety of NPs as well as identifying NPs that can be safely used as carriers of drugs targeting CNS related diseases. This screening is preferentially performed in insect models with intact BB function and this will contribute to a positive selection of NPs that are safe for vertebrates.

### SUMMARY OF THE INVENTION

The object of the present invention is to develop insect screening models to accurately assess both acute and chronic effects of nanoparticles on the function and integrity of the blood-brain barrier.

This object offers many advantages relative to prior technologies since insect models are more reliable tools for the decision-making process than the existing in vitro models, and will reduce the number of mammals sacrificed during the drug discovery phase.

Accordingly, there is provided a method of conducting studies of the effects of nanoparticles on the function and integrity of the blood-brain barrier, said method comprising the steps:
optionally anesthetizing the insect;
exposing the insect brain-barrier to the nanoparticle(s) of interest;
exposing the nanoparticle treated insect brain barrier to one or more suitable marker(s) of the function and integrity of the brain barrier
preparing the brain material; and
analyzing the effect of the NP(s) on the function and integrity of brain barrier by quantitative or qualitative measurements of the marker(s).

The method steps of the present invention may be varied with respect to when the insect is anesthetized and when the brain is eventually dissected out. Accordingly, the present invention also provides a method of conducting studies of the effects of nanoparticles on the function and integrity of the blood-brain barrier, said method comprising the steps:
treatment of an insect with test NPs;
exposing the nanoparticle treated insect to one or more suitable marker(s) of function and integrity of the brain barrier;
optionally anesthetizing an insect;
dissecting out the insect brain;
optionally removing the neural lamella, which is surrounding the BBB;
preparing the brain material; and
analyze the effect of the NP(s) on the function and integrity of brain barrier by quantitative or qualitative measurements of the marker molecules.

In still another embodiment the present invention provides a method of conducting studies of the effects of nanoparticles on the function and integrity of the blood-brain barrier, said method comprising the steps:
treatment of an insect with test NPs;
optionally anesthetizing an insect;
dissecting out the insect brain;
optionally removing the neural lamella, which is surrounding the BBB;
exposing the nanoparticle treated insect brain to one or more suitable marker(s) of function and integrity of the brain barrier;
preparing the brain material; and
analyze the effect of the NP(s) on the function and integrity of brain barrier by quantitative or qualitative measurements of the marker molecules.

In a preferred embodiment of the present invention NPs are coated with albumin to introduce the effect of plasma protein coating of the nanoparticles on the function and integrity of the brain barrier (free vs. protein coated nanoparticles).

In another preferred embodiment of the present invention NPs are coated with other coating molecules e.g. polysorbate, PEG or others to introduce the role of NP coating on the nanoparticle's effect on function and integrity of the brain barrier (free vs. specifically coated nanoparticles).

Preferable the concentration of the brain barrier function marker molecules permeating into the brain are determined by LC/MS or ICP-MS. In this respect the determination of the concentration of the marker molecule is performed by homogenizing or ultra sound disintegration of the dissected brains. The homogenate is centrifuged and the concentration of the test agent in the supernatant is then analyzed by liquid chromatography with mass spectrometric detection of the eluted compounds, by ICP-MS. In other studies the NPs effect is determined by using fluorescent molecule markers which are analyzed in brain slices by fluorescence microscopy or by fluorometry.

In order to ensure optimum effects of the NPs on barrier function the brain is exposed to the NP for a period of 1 min.-9 weeks. In a particularly preferred embodiment of the present invention the neural lamella of the brain is removed before the brain is exposed to the NP.

The method of the present invention permits the in vivo exposure to an insect brain of a NP in acute or chronic experiments. The method also permits the ex vivo exposure to an insect brain of NPs at stable concentrations during the entire period of exposure. The effect of the test NP on brain barrier function and integrity is measured by the use of selective molecular functional markers, which are analyzed by liquid chromatography with mass spectrometric detection of the eluted compounds, by ICP-MS or are determined by using fluorescent molecule markers which are analyzed in brain slices by fluorescence microscopy or by fluorometry.

Preferably the dissected brains are homogenized or disintegrated by ultra sound or other methods in order to obtain a homogenate reflecting the composition of the brains. The homogenate is centrifuged and the supernatant stored until analysis. The further analysis of the supernatant sample may be performed by virtue of liquid chromatography, possibly with mass spectrometric detection of the eluted compounds. Alternatively, the presence of the functional markers in the brain are indentified and quantified by histochemical methods (fluorescence microscopy or by fluorometry).

In various aspects and embodiments the present invention provides the subject-matter set out in the claims below.

### DETAILED DESCRIPTION OF THE INVENTION

The present invention provides a new methodology for screening the effects of nanoparticles on brain barrier function and integrity. Since the use of NPs has increased tremendously during the recent years and the NPs are widely spread also in the environment and there are great concerns for the potential effects on the function of living organisms as well as human beings. Certain NPs are taken up by the various barriers (e.g. lungs, intestinal mucosa or skin) and/or permeate the barriers including the brain barrier. Therefore, it is of utmost importance to identify NPs that are taken up by the brain barrier and affect the function and integrity of the barrier and provide experimental models for assessing the safety of the NPs.

The present invention provide an insect model that is generally useful for investigating the safety profile of NPs including NPs developed in drug discovery programs targeting a variety of diseases and disorders.

In preferred embodiments the NPs of the present invention is less than 100 nm, such as less than 50 nm diameter.

There are several methods for creating NPs, including both attrition and pyrolysis. In attrition, macro or micro scale particles are ground in a ball mill or other size reducing mechanism. Thermal plasma can also deliver the energy necessary to cause evaporation of small micrometer size particles. Inert-gas condensation is frequently used to make NPs from metals with low melting points. The metal is vaporized in a vacuum chamber and then super cooled with an inert gas stream. The super cooled metal vapor condenses into nanometer-sized particles, which can be entrained in the inert gas stream and deposited on a substrate or studied in situ.

The present invention relates to but is not restricted to the use of insects selected from the following orders: (Taxonomy according to: Djurens Värld, Ed B. Hanström; Förlagshuset Norden A B, Maömlö, 1964):

| Order | Suborder/family | Comment |
|---|---|---|
| Dictyoptera | Blattodea | Cockroach |
| | Mantoidea | |
| Orthoptera | Grylloidea | Crickets |
| | Acridoidea | Grasshoppers |
| Cheleutoptera | | Stick insects |
| Lepidoptera | | Moths |
| Hymenoptera | Formicoidea | Ants |
| | Vespoidea | Wasps |
| | Apoidea | Bee like |
| | | Hymenopterans |
| | Bombinae | Bumble-bees |
| | Apine | Proper bees |
| Odonata | | Dragonflies |
| Diptera | Nematocera | Mosquitos |
| | Brachycera | Flies E.g |
| | | Drosophila |

In particular the invention relates to insect species selected from Blattodea, Acridoidea, Cheleutoptera, Brachycera, Bombine, Apine and Lepidoptera and most particular to the Acridoidea (Locusta migratoria and Schistocerca gregaria).

The invention will also relate to the following orders comprising insect species relevant for the method of the present invention:

| Order | Suborder/family | Comment |
|---|---|---|
| Ephemerida | | Mayflies |
| Plecoptera | | |
| Dermoptera | Forficuloidea | Earwigs |
| Homoptera | Cicadinea | Cicadas |
| | Aphidine | Plant-louse |
| Heteroptera | | Hemipteran |
| Coleoptera | | Beetles |
| Trichoptera | | Caddis fly |

The present invention preferably uses large insects, such as the migratoty locust, Locusta migratoria and the desert locust, Schistocerca gregaria or cockroach where it is feasible to feed and inject drugs and subsequently take hemolymph samples and dissect brain tissues, for analyses. The locust has been used to develop screening models to determine NPs effect on blood-brain barrier function and integrity.

In accordance with a preferred embodiment of the present invention the migratoty locust, Locusta migratoria and/or the desert locust, Schistocerca gregaria, is used since it is easy to breed and it is a relatively large insect (40-60 mm long, weight: approx. 2 g, hemolymph volume: approx. 300 μL, brain weight: approx. 2 mg).

The exposure of nanoparticles to the insect brain barriers of the present invention in a screening method may be as follows, in accordance with a preferred embodiment of the present invention.

### Preferred Embodiments

In a preferred embodiment of the present invention the insects are selected from the order Acridoidea and specifically Locusta migratoria and Schistocerca gregaria are used. The insects may be obtained from professional breeders. The insects were reared under crowded conditions at 28°-38° and a 12:12 dark: light photo cycle. Animals used are adult males or females between one to six weeks after adult emergence or a maximal period of 9 weeks or during the whole life span of the grasshopper. After various times of exposure to the coated or non-coated NPs the effects of the NPs on brain barrier function and integrity is determined by using functional marker molecules. Preferably the brains are homogenized or disintegrated by ultra sound or other methods in order to obtain a homogenate reflecting the composition of the brains. The homogenate is centrifuged and the supernatant stored until analysis. The selective molecular functional markers are analyzed by liquid chromatography with mass spectrometric detection of the eluted compounds, by ICP-MS. Alternatively, the NPs effect on the barrier function and integrity is obtained by using fluorescent functional markers. The presence of these markers in the brain are identified and/or quantified in brain slices by using fluorescence microscopy or by fluorometry.

In a preferred embodiment of the present invention the insects are selected from the order Acridoidea and specifically Locusta migratoria and Schistocerca gregaria are used. The insects may be obtained from professional breeders. Animals used are adult males or females between one to six weeks after adult emergence or a maximal period of 9 weeks or during the whole life span of the grasshopper. After various times after in vivo administration (oral, tracheal or intrahemolymphic) the effects of the coated or non-coated NPs on brain barrier function and integrity is determined by using functional marker molecules injected into the hemolymph at end of the exposure period. The selective molecular functional markers are analyzed by liquid chromatography with mass spectrometric detection of the eluted compounds, by ICP-MS or are determined by using fluorescent molecule markers which are analyzed in brain slices by fluorescence microscopy or by fluorometry.

In a preferred embodiment of the present invention the insects are selected from the order Acridoidea and specifically Locusta migratoria and Schistocerca gregaria are used. The insects may be obtained from professional breeders. Animals used are adult males or females between one to six weeks after adult emergence or a maximal period of 9 weeks or during the whole life span of the grasshopper. After various times after in vivo administration (oral, tracheal or intrahemolymphic) the insect brains are dissected out. The effects of the coated or non-coated NPs on brain barrier function and integrity is determined by using functional marker molecules, which are exposed ex vivo to the dissected insect brains. The selective molecular functional markers are analyzed by liquid chromatography with mass spectrometric detection of the eluted compounds, by ICP-MS or are determined by using fluorescent molecule markers which are analyzed in brain slices by fluorescence microscopy or by fluorometry.

In a preferred embodiment of the present invention the insects are selected from the order Acridoidea and specifically Locusta migratoria and Schistocerca gregaria are used. The insects may be obtained from professional breeders. Animals used are adult males or females between one to six weeks after adult emergence or a maximal period of 9 weeks. A cut is made through the frontal part of the locust head comprising the most frontal parts including the antennae, the compound eyes, the brain and all neural connections between the brain and the antennae and the eyes. The brain is dissected out and placed in a well of a microtitre plate containing the coated or non-coated NP. After various times of exposure the brain is washed in cold insect buffer and the neural lamella surrounding the brain is removed. The effects of the NPs on brain barrier function and integrity is determined by using functional marker molecules added to the incubation well at end of the exposure period. The selective molecular functional markers are analyzed by liquid chromatography with mass spectrometric detection of the eluted compounds, by ICP-MS or are determined by using fluorescent molecule markers which are analyzed in brain slices by fluorescence microscopy or by fluorometry.

In a preferred embodiment of the present invention the insects are selected from the order Acridoidea and specifically Locusta migratoria and Schistocerca gregaria are used. The insects may be obtained from professional breeders. Animals used are adult males or females between one to six weeks after adult emergence or a maximal period of 9 weeks. A cut is made through the frontal part of the locust head comprising the most frontal parts including the antennae, the compound eyes, the brain and all neural connections between the brain and the antennae and the eyes. The brain is dissected out, the neural lamella removed and this preparation is placed in a well of a microtitre plate containing the coated or non-coated NP. After various times of exposure the brain is washed in cold insect buffer. The effects of the NPs on brain barrier function and integrity is determined by using functional marker molecules added to the incubation well at end of the exposure period. The selective molecular functional markers are analyzed by liquid chromatography with mass spectrometric detection of the eluted compounds, by ICP-MS or are determined by using fluorescent molecule markers which are analyzed in brain slices by fluorescence microscopy or by fluorometry.

### EXAMPLE

Locust brains were dissected in insect buffer, placed in solutions containing fluorescent polystyrene amino modified 100 nm NPs and exposed for 3 hours. The brains were washed in cold insect buffer, the neural lamella removed and the brains were then exposed to Evans blue for one minute and then analyzed for dye uptake. There was no significant uptake of Evans blue in the locust brain barrier.

Locust brains were dissected in insect buffer and the neural lamella removed. The brains were placed in solutions containing fluorescent polystyrene amino modified 100 nm NPs and exposed for 3 hours. The brains were washed in cold insect buffer and then exposed to Evans blue for one minute and then analyzed for dye uptake. There was a marked uptake of Evans blue in the locust brain barrier.

Locust brains were dissected in insect buffer, placed in solutions containing fluorescent polystyrene amino modified 50 nm NPs and exposed for 3 hours. The brains were washed in cold insect buffer, the neural lamella removed and the brains were then exposed to Evans blue for one minute and then analyzed for dye uptake. There was no uptake of Evans blue in the locust brain barrier.

Locust brains were dissected in insect buffer and the neural lamella removed. The brains were placed in solutions containing fluorescent polystyrene amino modified 50 nm NPs and exposed for 3 hours. The brains were washed in cold insect buffer and then exposed ex vivo to Evans blue for one minute and then analyzed for dye uptake. There was no uptake of Evans blue in the locust brain barrier.

Locust brains were dissected in insect buffer, placed in solutions containing BSA coated silver NPs (80 nm) and exposed for 3 hours. The brains were washed in cold insect buffer, the neural lamella removed and the brains were then exposed to Evans blue for one minute and then analyzed for dye uptake. There was a highly significant uptake of Evans blue in the locust brain barrier.

Locust brains were dissected in insect buffer and the neural lamella removed. The brains were placed in solutions containing BSA coated silver NPs (80 nm) and exposed for 3 hours. The brains were washed in cold insect buffer and then exposed ex vivo to Evans blue for one minute and then analyzed for dye uptake. There was a highly significant uptake of Evans blue in the locust brain barrier.

Polystyrene NPs (100 nm) were injected into the hemolymph of locusts. After 24 hours the locust brains were dissected in insect buffer, the neural lamella removed and the brains washed in cold insect buffer. The brains were treated with Evans blue for 1 minute and then analyzed for dye uptake. There was no uptake of Evans blue in the locust brain barrier.

Polystyrene NPs (50 nm) were injected into the hemolymph of locusts. After 24 hours the locust brains were dissected in insect buffer, the neural lamella removed and the brains washed in cold insect buffer. The brains were treated with Evans blue for 1 minute and then analyzed for dye uptake. There was no uptake of Evans blue in the locust brain barrier.

Silver NPs (57 nm) were injected into the hemolymph of locusts. After 24 hours the locust brains were dissected in insect buffer, the neural lamella removed and the brains washed in cold insect buffer. The brains were treated with Evans blue for 1 minute and then analyzed for dye uptake. There was a marked uptake of Evans blue in the locust brain barrier.

Conclusion: The ex vivo locust model clearly shows that different types of NPs differently affect the function of the locust brain barrier. The ex vivo model, without the neural lamella, differentiate the polystyrene NPs depending on size which may be related to a size dependent induction of particle incorporation of the barrier cells. Silver NPs affected the barrier function irrespective of the absence or presence of the neural lamella.

The in vivo model also showed discrimination between the NPs. Silver NPs affected the barrier function whereas there were no effects of polystyrene NPs.

### REFERENCES

Abbott, N. J. 2005 Dynamics of CNS barriers: evolution, differentiation, and modulation. Cell Mol Neurobiol 25: 5-23
Daneman, R. and Barres, B. A. 2005 The blood-brain barrier; lessons from moody flies. Cell 123: 9-12
Garberg, P. et al. (2005). In vitro models for the blood-brain barrier. Toxicology in Vitro 19, 299-334.
Johnston, H. J. et al., 2010. A review of the in vivo and in vitro toxicity of silver and gold particulates: Particle attributes and biological mechanisms responsible for the observed toxicity. Critical Reviews in Toxicology, 40(4): 328-346.
JRC Reference Report (EUR 24847 EN). Impact of engineered nanomaterials on health: Considerations for benefit-risk assessment. Joint EASAC-JRC Report September 2011.
Lai, D. Y. (2011). Toward toxicity testing of nanomaterials in the 21^{st} century: a paradigm for moving forward. WIREs Nanomed Nanobiotechnol DOI: 10.1002/wnan.162.
Mayer, F. et al., 2009 Evolutionary conservation of vertebrate blood-brain barrier chemoprotective mechanisms in Drosophila. J Neuroscience 29: 3538-3550.
Nic Ragnaill, M. Et al., (2011). Internal benchmarking of a human blood-brain barrier cell model for screening of nanoparticle uptake and transcytosis. Eur. J. Pharm. Biopharm. In press.
Nielsen, P. A. et al., (2011). Models for predicting blood-brain barrier permeation. Drug Discov. Today. 16(11-22): 472-475.
Pardridge, W. M. (2002). Drug and gene targeting to the brain with molecular Trojan horses. Nature Reviews Drug Discovery 1, 131-139
Pardridge W. M., 2005 Molecular biology of the blood-brain barrier. Mol Biotechnol 30: 57-70.
dos Santos, T. Et al., (2011). Quantitative assessment of the comparative nanoparticle uptake efficiency of a range of cell lines. Small; 23: 3341-3349.
Posgai, R. et al., (2009). Inhalation method for delivery of nanoparticles to the Drosophila respiratory system for toxicity testing. Sciene of the Total Environment. 408: 439-443.
Schinkel, A. H. (1999). P-Glycoprotein, a gatekeeper in the blood-brain barrier. Advanced Drug Delivery Reviews 36, 179-194.
Stern, S. T. and McNeil, S. E. (2008). Nanotechnology safety concerns revisited. Toxicological Sciences 101(1): 4-21.
Stone, V. Et al., (2009). Development of in vitro systems for nanotoxicology: methodological considerations. Critical Reviews in Toxicology. 39(7): 613-626.
Xia, C. Q., Xiao, G., Liu, N., Pimprale, S., Fox, L., Patten, C. J., Crespi, C. L., Miwa, G., Gan, L.-S. (2006). Comparison of Species Differences of P-Glycoproteins in Beagle Dog, Rhesus Monkey, and Human Using ATPase Activity Assays. Molecular Pharmaceutics 3 (1), 78-86.
Wu V M and Beitel G J, 2004 A junctional problem of apical proportions: epithelial tube-size control by septate junctions in the Drosophila tracheal system. Curr Opin Cell Biol 16: 493-499.
Zlokovic, B. V. 2008 The blood-brain barrier in health and chronic neurodegenerative disorders. Neuron 57: 178-201.

1. A method of conducting studies of the effects of nanoparticles on the function and integrity of the blood-brain barrier, said method comprising the steps:
treatment of an insect with test NPs;
optionally anesthetizing an insect;
dissecting out the insect brain;
optionally removing the neural lamella, which is surrounding the BBB;
exposing the nanoparticle treated insect brain to one or more suitable marker(s) of function and integrity of the brain barrier;
preparing the brain material; and
analyze the effect of the NP(s) on the function and integrity of brain barrier by quantitative or qualitative measurements of the marker molecules.

2. Method according to claim 1, wherein the nanoparticles are coated.

3. Method according to claim 2, wherein nanoparticles are coated with albumin to introduce the effect of plasma protein coating of the nanoparticles.

4. Method according to claim 2, wherein nanoparticles are coated with polysorbate or PEG or other coating materials.

5. Method according to claim 1, wherein the quantitative measurements are determined by LC/MS(-MS) or ICP-MS or by fluorometry.

6. Method according to claim 1, wherein the determination of the marker molecule is performed by homogenizing or ultra sound disintegrating the dissected brains, followed by centrifugation and quantitative measurements are determined by LC/MS(-MS) or ICP-MS or by fluorometry.

7. Method according to claim 1, wherein the NPs marker molecule is a fluorescent molecule, which can be analyzed in brain slices by fluorescence microscopy.

8. Method according to claim 1, wherein the step of exposing the insect brain-barrier to the nanoparticle(s) of interest lasts for a period of 1 min.-9 weeks or throughout the whole life-span of the locust.

9. Method according to claim 1, wherein the neural lamella of the brain is removed before the brain is exposed to the NP.
